(19) United States
(12) Patent Application Publication
(10) Pub. No.: US 2005/0002969 A1
(43) Pub. Date: Jan. 6, 2005

(54) Binary composition for prime-boost release of active ingredients like vaccines

(76) Inventor: Glenn Patrick Martyn, Nottingham (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/492,055

(22) PCT Filed: Oct. 15, 2002
(86) PCT No.: PCT/GB02/04664

(30) Foreign Application Priority Data
Oct. 15, 2001 (GB) 0124710.5

(51) Int. Cl. A61K 39/02; A61K 31/739

(52) U.S. Cl. 424/234.1; 514/54

(57) ABSTRACT

Therapeutic compositions in solid dose form that comprise a mixture of first amorphous or non-crystalline microparticles comprising a bioactive agent and second amorphous or non-crystalline microparticles that comprise the same or different bioactive agent. The compositions provide a primary pharmacological response (first microparticles) and a second “boosting” effect (second microparticles) produced by releasing the agent over a linger period.

### FIELD OF THE INVENTION

This invention relates to therapeutic compositions, and in particular to vaccines of the “prime-boost” type.

### BACKGROUND OF THE INVENTION

“Prime-boost” formulations typically comprise an immunogen formulated in two ways, the first to give a priming dose and the second to give a boost. This can be an effective system for the delivering of immunogens, in order that the subject is effectively immunised.

While the “prime-boost” vaccine concept is known, developments have been few, although relatively advanced in areas such as HIV, infectious diseases, cancer and naked DNA vaccines. The efficacy of DNA-based vaccines has been greatly enhanced by boosting with live recombinant virus vaccines. The two components are often given separately and chronologically apart. It would be advantageous to have a unit dose delivery system, whereby fewer individual injections need to be administered.

For example, with regard to anthrax, the immunisation programme consists of a series of six innoculations over a 18 month period. It would be beneficial to avoid or reduce this.

Solid delivery systems for controlled release are described in WO-A-9603978 (the content of which is incorporated herein by reference). Such compositions comprise an active agent and a glassy vehicle composed of a stabilising polymer or hydrophobic derivatised carbohydrate (HDC).

EP-A0678035 discloses a vaccine preparation in a controlled-release formulation. The vaccine is prepared by spray drying an immunogen adsorbed to an aluminium salt adjuvant, to form a free-flowing powder. The vaccine is then administered to a patient in the form of a liquid suspension via the parenteral route. In one embodiment, the vaccine composition comprises at least one immediate-release vaccine preparation and at least one controlled-release vaccine preparation. The controlled-release preparation is formulated using a biodegradable polymer, including polyesters, polyanhydrides, cyanoacrylates and homopolymers of polylactic acids.

The need to form the liquid suspension for injection is problematic, especially if the vaccine is to be used in remote regions, where sterile water is difficult to prepare.

### SUMMARY OF THE INVENTION

According to a first aspect of the present invention, a therapeutic composition in solid dose form comprises a mixture of first amorphous or non-crystalline microparticles comprising a bioactive agent and second amorphous or non-crystalline microparticles comprising the same or a different bioactive agent. The first microparticles provide a primary pharmacological response and the second microparticles provide sustained, delayed or pulsatile release of the agent contained therein over a longer period. For example, the release of the agent may be over (or may be delayed for) days, weeks or months. An alternative option is that the primary response is provided by the bioactive agent in a different form.

The present invention utilises the properties of the variety of glassy vehicles that can be produced, having different release characteristics. In particular, controlled release can be achieved using a HDC, preferably in a sustained, delayed or pulsatile manner.

In a preferred aspect, the present invention provides a single dose stabilised vaccine which contains glassy microparticles comprising the same or different antigens, whereby first microparticles present the antigen rapidly (a so-called “priming” effect) and second microparticles present the antigen in a controlled manner (sustained, delayed or pulsatile manner) over a protracted time period (“boost” effect).

According to a further aspect of the invention, a device for delivering a bioactive agent to a patient comprises a composition described above.

### DESCRIPTION OF THE INVENTION

The present invention makes use of known products to formulate the first and second microparticles, to achieve the “prime-boost” effect.

Therapeutic compositions of the invention are said to be in “solid dose” form. The compositions are therefore solids, not solutions. Although the preferred embodiment is a dry powder composition, where the first and second microparticles are administered in this form, the invention also contemplates the presentation of the microparticles in an aqueous or non-aqueous medium for subsequent delivery. The microparticles will preferably be solids in suspension.

The microparticles are defined as “amorphous or non-crystalline”. Those terms are familiar in the art, arid methods for establishing whether a structure is amorphous or non-crystalline are known. For example optical microscopy can be used, as will be appreciated by the skilled person.

In one embodiment of the invention, a rapidly devitrifying HDC is used as the “primer” vehicle and a slower or non-vitrifying HDC is used as the controlled release (CR) matrix for the “booster” fraction. Suitable HDCs (including TOAc, i.e. trehalose octaacetate) are described in WO-A-9603978, WO-A-9829097 and WO-A-9933853, the content of each being incorporated herein by reference.

Alternatively, the vehicle of the primer particles may be a stabilising polyol (SP). The booster fraction may also be SP-based but further contain a CR glass, such as PLA/PLGA, etc. Again, such components are described in WO-A-9603978. For example, suitable stabilising polyols include carbohydrates. The carbohydrates include monosaccharides, disaccharides, oligosaccharides and their corresponding sugar alcohols. Typically the SP will have a glass transition temperature (Tg) greater than 30° C., preferably greater than 40° C. and more preferably greater than 50° C. Preferred SPs include trehalose, sucrose and raffinose.

In a further embodiment, a SP-based primer fraction may be mixed with a CR HDC-based fraction, to elicit the same effect. Conversely, the rapid release fraction may be HDC-based, e.g. TOAc, whilst the booster microparticles comprise SP/PLA/PLGA, etc.

Such a blend may be delivered as a unit dose via various routes of administration (see WO-A-9603978 for illustrative examples). This vaccine delivery format may also provide an additive or synergistic immune response. It may also provide systemic and mucosal immunity. Thus, the invention can take advantage of the fact that the route of entry for many pathogens is by way of mucosal surfaces, and immunity at such sites can limit or even prevent infection. There is also evidence that the mucosal immune system is inter-linked whereby, following mucosal immunisation, immunity is evident at a mucosal site some distance from the actual site of administration. Thus, pulmonary administration of say, a herpes virus vaccine, may provide vaginal mucosal defence against the sexually-transmitted form of the disease.

Such a prophylactic delivery system may provide a “prime-boost” effect. However, a similar “load-sustain” pharmacological response may be achieved from such a delivery system comprising a therapeutic bioactive.

The first and second microparticles may be of any suitable size. Preferably, the microparticles are from 0.1 μm to 100 μm in diameter. The first and second microparticles may be the same or different sizes.

One advantage of the prime-boost method is its potential to induce at least additive immune responses. For instance, the simultaneous or subsequent administration of subunit antigens (boost) with pox-based vaccines (prime) results in complementary immune responses that include the induction of CTL activity, neutralising antibody, proliferative responses (an indicators of T-cell help) and antibody-dependent cytotoxic activity (ADCC). Further, memory T-lymphocytes can mobilise rapidly and clone themselves if a specific antigen, encountered during infection or vaccination, appears at a later time.

Multivalent vaccines may be prepared by presenting more than one antigen in the same primer microparticles or by simply delivering a mixture of priming microparticulates. The converse may also apply to the booster microparticulate fraction.

A formulation of the invention that comprises an immunogen preferably also comprises an adjuvant. An adjuvant effect may be provided by a HDC. Examples of HDCs having different dissolution rates in vivo are TOAc and trehalose octapivalate and the adjuvant effect is related to their relative insolubility.

Other suitable adjuvants include, but are not limited to, aluminium salts, squalene mixtures, muramyl peptide, saponin derivatives, mycobacterium cell wall preparations, immunostimulating complexes (ISCOMs) and nonionic block copolymer surfactants. For veterinary use, mitogenic components of Freund's adjuvant can be used.

The compositions of the invention may be adapted for any suitable route of administration, including sub-cutaneous, intra-venous, intra-dermal, intra-muscular, intra-ocular and intra-peritoneal. In a preferred embodiment, the compositions are adapted for mucosal delivery, and delivered to the patient using known dry powder and liquid delivery systems (e.g. nebulisers and pMDI). Suitable dry powder inhalation devices are known in the art. In this context, the compositions are formulated as dry powders, and may include suitable carriers as is known in the art. For example, sugars, including lactose and mannitol having a particle size of from 25 μm to 500 μm, preferably 50 μm to 250 μm in diameter are known in the art. Other aerosol devices requiring perfluorocarbons may also be used.

Alternative devices include needle-less injections including ballistic dry powder devices and liquid needle-less injection devices.

Mucosal delivery includes delivery via inhalation, (nasal, trans-alveolar and trans-bronchial), rectal and vaginal.

The compositions may be formulated to include other components that aid mucosal delivery. For example, mucoadhesive agents, including cellulose and its derivatives, starch, carbopol, poloxamers, chitosan and its derivatives and hyaluronic acid, may be incorporated into or around the microparticles, to aid administration via the mucosal route.

Absorption enhancing materials may also be present. Suitable materials include, phospholipids, chelating agents, mucolytics, peptide inhibitors, and surface active agents selected from the group consisting of bile salts, fatty acids, fatty acid salts, acylglycerols, tyloxapols, acylcarnitine, fusidates, and mixtures thereof.

Although described above with reference to vaccines, the microparticles may be formulated with any suitable bioactive agent. The term “bioactive” is intended to include any pharmacologically active agent, useful for treatment or prophylaxis. Example bioactive agents include, but are not limited to, peptides or proteins, hormones, analgesics, anti-migraine agents, anti-coagulant agents, narcotic, antagonists, chelating agents, anti-anginal agents, chemotherapy agents, sedatives, compounds for the treatment of Alzheimer's disease (amyloid β or fragments thereof), anti-neoplastics and cardiovascular drugs. Preferred bioactive agents include insulin, erythropoietin (EPO), interferons (α, β or γ), somatrotropin, somatostatin, tissue plasminogen activator (TPA), anti-malarial compounds, growth hormone releasing hormone, factor VIII and interleukin. As stated previously, immunogens are particularly preferred. The same or different antigens may be present in the microparticles. The immunogens may be used in the prophylaxis of any bacterial o viral disease. For example, the immunogen may be for the prevention of meningococcal disease (meningitis, septicaemia, meningoccaemia and pneumonia). In this embodiment, the immunogen may be used to prevent infection of meningococci of any of groups A, B, C, Y, W135, X and Z.

Other suitable immunogens for use in the practice of the invention include vaccines against anthrax (protective antigen), plague, small pox, tularaemia, meliodosis, Q fever, botulism, typhus, cholera, yellow fever, brucellosis, encephalitis, ricin, salmonella and staphylococcal Enterotoxin B.

Viral particles useful in the preparation of vaccines are known and are applicable to the invention. The invention may be used for the prophylaxis of HIV, HepB, CMV and TB.

In an alternative embodiment of the invention, a unit dose inhalation powder, for therapy of diabetes, comprises a rapid-acting insulin fraction (pure or stabilised in a trehalose glass) together with a CR fraction comprising HA (or HA/HPC or Zn-complexed insulin embedded in HA). In this way, a meal-time insulin dose may be provided by the rapidly soluble component with basal plasma insulin being provided via the CR fraction. This may be applicable to a number of delivery routes and with the same excipient formats described above.

The following Examples illustrate the invention.

### EXAMPLE 1

A dry powder blend of first and second microparticles is prepared as follows:

Prime Fraction

Microparticles are prepared by spray drying a formulation comprising HepB antigen and Trehalose using a Buchi 191 Mini Laboratory Spray Dryer. The resulting microparticles have a particle size of less than 5 μm.

Boost Fraction

Microparticles are prepared by spray drying a formulation comprising HepB antigen and the HDC Di-(β-Tetraacetyl Glucurnoyl) Hexaacetyl Trehalose (prepared according to International Application No. PCT/GB01/04932) using a Buchi 191 Mini Laboratory Spray Dryer. The resulting microparticles have a particle size of less than 3 μm, with the majority of the particles having a distribution between 1 to 2 μm.

The microparticles of the prime fraction and boost fraction are then blended to form one unit dose, and filled into a blister pack for subsequent administration via a dry powder inhaler device.

### EXAMPLE 2

A dry powder blend of microparticles is prepared as follows.

Immediate Release Formulation

A solution of 20% (w/v) zinc insulin and 80% (w/v) Trehalose is prepared and spray dried using the Buchi Mini Laboratory Spray Dryer.

Microparticles are produced having a size less than 5 μm in diameter.

Controlled Release Formulation

A formulation containing 25% w/w HPC (ex. Nippo Soda Co., Japan) 10% w/w recombinant human insulin and 65% w/w high molecular weight hyaluronic acid (ex. Genzyme) is prepared as follows. To 154 mg insulin is added 2.16 ml 0.05M HCl and swirled gently until dissolved. To this solution is added dropwise 0.14 ml 1M NaOH together with 165 ml purified water. This solution is then added to 96.25 ml of 0.4% w/v HPC solution and then 250 ml of a 0.4% w/v solution of high molecular weight hyaluronic acid is added and the mixture stirred until homogenous. Approximately 500 ml of this feedstock is spray dried at the following settings: feed rate=2.1 g/min, inlet temp=130° C., outlet temp=66° C., atomisation=2-fluid nozzle, atomisation pressure=2 barg, atomisation airflow rate=21 l/min, drying air pressure=1 barg, drying air flow rate=5 l/sec.

The two formulations are then blended together to form the dual release system.

1. A therapeutic composition in solid dose form comprising a powder blend of first amorphous or non-crystalline microparticles comprising a bioactive agent and second amorphous or non-crystalline microparticles comprising the same or a different bioactive agent, said first microparticles providing a primary pharmacological response and said second microparticles providing release of the agent contained therein over a longer period.

2. The composition according to claim 1, wherein the or each bioactive agent is an immunogen.

3. The composition according to claim 2, wherein the immunogen is an attenuated bacteria, or an immunogenic peptide/protein obtainable from a bacterium.

4. The composition according to claim 2, wherein the immunogen is a viral particle.

5. The composition according to claim 1, wherein the first bioactive agent and/or the second microparticles comprise an adjuvant.

6. The composition according to claim 1, wherein the bioactive agents are the same.

7. The composition according to claim 1, wherein the second microparticles comprise, as a vehicle, a stabilizing polyol.

8. The composition according to claim 1, wherein the second microparticles comprise a HDC.

9. The composition according to claim 1, wherein the first and second microparticles are from 0.1 μm to 100 μm in diameter.

10. The composition according to claim 1, wherein the first and second microparticles are from 0.5 μm to 3 μm in diameter.

11. The composition according to claim 1, wherein the first microparticles comprise, as a vehicle, a stabilizing polyol or a hydrophobic derivatised carbohydrate (HDC).

12. The composition according to claim 11, wherein the first microparticles comprise a stabilizing polyol.
